(19) United States
(12) Patent Application Publication
(10) Pub. No.: US 2004/0267099 A1
(43) Pub. Date: Dec. 30, 2004

(54) Pain assessment user interface

(76) Inventors: Michael D. McMahon, Lake Forest Park, WA (US); Christopher Pearce, Monroe, WA (US); John C. Daynes, Redmond, WA (US)

Correspondence Address: SHUMAKER & SIEFFERT, P. A., 8425 SEASONS PARKWAY, SUITE 105, ST. PAUL, MN, 55125, US

(21) Appl. No.: 10/610,375

(22) Filed: Jun. 30, 2003

(51) Int. Cl. A61B 5/00; A61B 19/00

(52) U.S. Cl. 600/300; 128/897; 128/898

(57) ABSTRACT

In general, the invention is directed to a user interface for patient pain assessment. The user interface allows an operator to input and store a patient's pain assessment based on a given pain assessment scale. The invention may tranmit the pain assessment data along with patient physiological condition data to a remotely located hospital database. As part of the patient's medical record, pain assessment measurements can provide trending information similar to most physiological condition data, which may be useful for future treatment. The invention may prompt the operator with suggestions for treatment based on the pain assessment and physiological data collected. In some embodiments, the user interface may be applied to a defibrillator or a patient monitor.

### TECHNICAL FIELD

The invention relates to medical devices and, more particularly, to emergency medical devices including event recording capabilities.

### BACKGROUND

Tracking physiological conditions, including heart rate, blood pressure, temperature, and the like, provides data that is important in diagnosing and treating a patient. The amount of pain that the patient is experiencing is also an important value when determining the best course of action to treat the injury or disease. For example, one method of treatment may be selected over another method if it is believed that the patient could handle an increase level of pain based on an initial pain assessment. An incorrect diagnosis may be avoided if the reported level of pain does not fit the usual symptoms. In particular, the pain assessment may cause a medical technician to reevaluate an initial assumption. Patients tend to have different tolerances to injuries and medication that only they can determine. Consequently, asking a patient to specify a level of pain may ensure that the patient is kept as comfortable as possible without being overmedicated. Pain assessment is also used to determine if a drug treatment, such as morphine or nitroglycerin, is an effective therapy by comparing the patient's pain levels over a period of time.

Patient pain assessment is very subjective and difficult to quantify, because it relies on the patient's judgment. This makes it difficult to compare pain levels between patients with similar diagnoses. In order to create a more definitive pain measurement technique, some devices generate a stimulus that the patient can compare to a perceived level of pain. For Example, a small voltage may be applied to the patient's skin. The patient is instructed to signal when the pain from the stimulus is equivalent to the pain due to the aliment or injury. This technique can determine a pain baseline for each patient and allow a more mathematical approach to the pain measurement process.

In general, existing pain assessment techniques comprise a simple numerical scale starting with zero or one being representative of no or little pain and ending with five or ten being representative of most pain possible. The numerical scale may be accompanied by pictorial representations of the pain levels, such as facial expressions. The facial expression scale uses simple line drawings to indicate the increasing distress and discomfort associated with an increase in pain. The patient is presented with the scale either verbally or physically. The patient responds by speaking the number or pointing to the facial expression that best describes the level of pain. This simple technique may not be very useful when comparing pain levels between patients. On a case by case basis, however, this technique can give the medical staff an idea of what the patient is experiencing.

### SUMMARY

In general, the invention is directed to a user interface for patient pain assessment. The user interface may be integrated with a medical therapy or diagnosis device, and allows an operator to input and store a patient's pain assessment based on a given pain assessment scale. As part of the patient's medical record, pain assessment measurements can provide trending information similar to other physiological condition data, which may be useful for future treatment. In addition, the pain assessment measurements can form a valuable part of an information record, such as run report, that documents various information during the course of a medical treatment or monitoring episode. In some embodiments, the user interface may be incorporated within a defibrillator or a patient monitor.

In one embodiment, the invention is directed to a method in which a patient pain assessment is received from an operator and patient physiological condition data is received from a physiological condition detector. This information is then stored in a memory. The pain assessment data may be transmitted along with patient physiological condition data to a remotely located hospital database to include in the patient's medical record. The patient pain assessment is based on a pain assessment scale that may be presented to the operator. The patient physiological condition may include blood pressure, blood oxygen saturation, body temperature, cardiac rhythm, respiration rate, and the like.

In another embodiment, the invention is directed to a device that includes a first input device and a second input device. The first input device is used by an operator to enter a patient pain assessment that is based on a pain assessment scale. The second input device includes a physiological condition detector used to gather patient physiological condition data that may include blood pressure, blood oxygen saturation, body temperature, respiration rate, and the like. The device also includes a memory to store the information collected by the first and second input devices. The invention may also include an output to enable operator prompts to suggest treatments based on the pain assessment and physiological data input.

In another embodiment, the invention is directed to a computer-readable medium containing instructions executable by a processor. The instructions cause a processor to receive a patient pain assessment from an operator interface, receive patient physiological condition data from a physiological condition detector, and record the patient pain assessment and the physiological data within a memory. The instructions may also cause a processor to create a report of the information stored in the memory. In some embodiments, the instructions may cause a processor to react to patient pain assessment input with output prompts providing further information to the operator.

The invention may provide one or more advantages. For example, unlike conventional pain assessment techniques, the pain assessment user interface creates an electronic record of the entered pain level and may be combined with other physiological data. The data gathered may be transmitted to a remotely located hospital database to become part of the patient's medical record, so the pain assessments may be used to determine a best course of action for the patient in the future. Also, the invention may prompt the operator with suggestions for treatment based on the pain assessment and physiological data collected.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an external defibrillator suitable for use with a pain assessment user interface in accordance with one embodiment of the invention.

FIG. 2 is a block diagram illustrating an example patient monitor suitable for use with a pain assessment user interface in accordance with another embodiment of the invention.

FIG. 3 is a block diagram illustrating an example of a defibrillator or patient monitor display presenting a patient pain assessment user interface.

FIG. 4 is a block diagram illustrating an example of a defibrillator or patient monitor display presenting a patient pain assessment user interface.

FIG. 5 is a block diagram illustrating an example of a defibrillator or patient monitor display using another patient pain assessment user interface.

FIG. 6 is a block diagram illustrating an example of a defibrillator or patient monitor display using another patient pain assessment user interface.

FIG. 7 is a block diagram illustrating an example of a defibrillator or patient monitor display using another patient pain assessment user interface.

FIG. 8 is a block diagram illustrating an example of a defibrillator or patient monitor display using another patient pain assessment user interface.

FIG. 9 is a block diagram illustrating an example of a defibrillator or patient monitor display using another patient pain assessment user interface.

FIG. 10A is a block diagram conceptually illustrating a report from an example patient record.

FIG. 10B is a block diagram conceptually illustrating another report from an example patient record.

FIG. 10C is a block diagram conceptually illustrating another report from an example patient record.

FIG. 11 is a block diagram illustrating an interaction between a patient and a defibrillator operator using a pain assessment user interface.

FIG. 12 is a flow chart illustrating a method for using a patient pain assessment user interface.

FIG. 13 is a flow chart illustrating a method for using a patient pain assessment user interface in greater detail.

### DETAILED DESCRIPTION

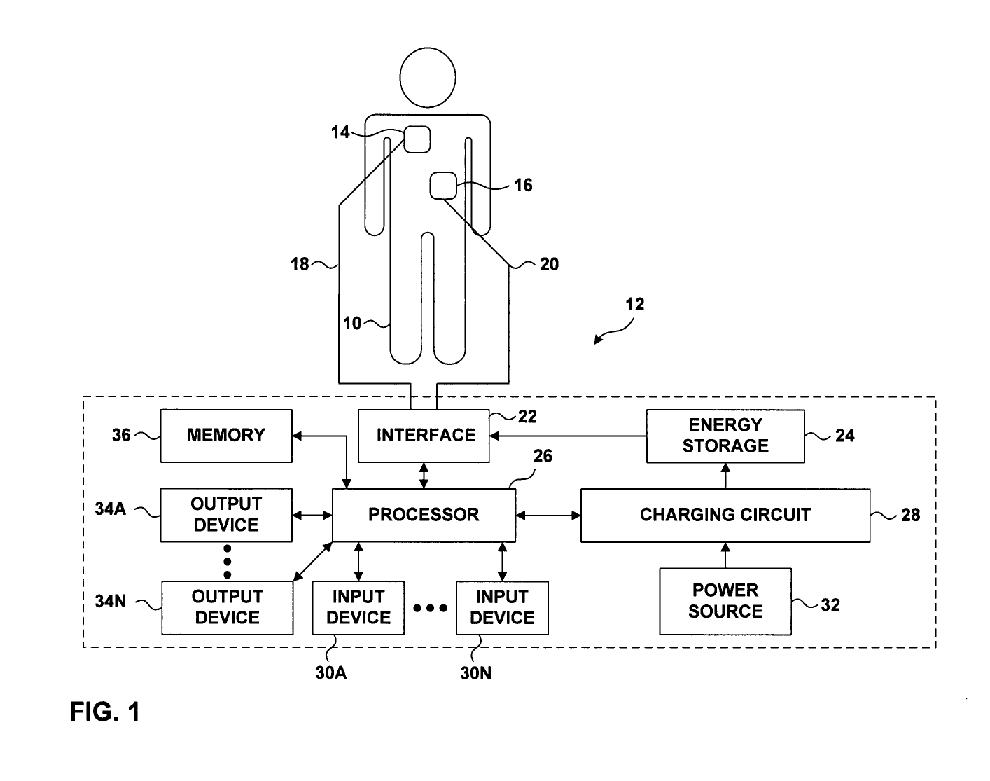

FIG. 1 is a block diagram illustrating an external defibrillator 12 suitable for use with a pain assessment user interface in accordance with one embodiment of the invention. As shown in FIG. 1, defibrillator 12 is coupled to a patient 10 by an electrode 14 and an electrode 16, which may be hand-held electrodes paddles or adhesive electrodes pads placed on the skin of patient 10. Electrodes 14 and 16 are coupled to defibrillator 12 by a conductor 18 and a conductor 20, respectively. Defibrillator 12 also includes a stimulation interface 22, an energy storage circuit 24, a processor 26, a charging circuit 28, one or more input devices 30A-30N (hereinafter 30), a power source 32, one or more output devices 34A-34N (hereinafter 34), and a memory 36.

Conductors 18 and 20 are coupled to interface 22. In a typical application, interface 22 includes a receptacle (not shown), and conductors 18 and 20 plug into the receptacle. Interface 22 may also include a switch that, when activated, couples energy storage circuit 24 to electrodes 14 and 16 to deliver stimulation energy in the form of a defibrillation shock.

Energy storage circuit 24 includes components, such as one or more capacitors, which store the energy to be delivered to patient 10 via electrodes 14 and 16 as a defibrillation shock. Before a defibrillation shock may be delivered to patient 10, energy storage circuit 24 must be charged. Processor 26 directs charging circuit 28 to charge energy storage circuit 24 to a voltage level determined by processor 26. Processor 26 may determine the voltage level based on a defibrillation shock energy level that may be, for example, input by an operator via input device 30, or selected by processor 26 from a preprogrammed progression of defibrillation shock energy levels stored in memory 36.

Processor 26 may activate the switch within interface 22 to cause delivery of the energy stored in energy storage circuit 24 across electrodes 14 and 16. Processor 26 may modulate the defibrillation shock delivered to patient 10. Processor 26 may, for example, control the switch to regulate the shape of the waveform of the shock and the width of the shock. Processor 26 may control the switch to modulate the shock to, for example, provide a multiphasic pulse, such as a biphasic truncated exponential pulse, as is known in the art. Processor 26 may take the form of a microprocessor, digital signal processor (DSP), application specific integrated circuit (ASIC), field-programmable gate array (FPGA), or other logic circuitry programmed or otherwise configured to operate as described herein.

Output devices 34 may include a display screen, a touch screen, an indicator light, a speaker, or the like. Processor 26 may display instructions to the operator via the display screen or the touch screen, and an electrocardiogram (ECG) and heart rate of patient 10 monitored by electrodes 14 and 16 may also be displayed via the screens. Defibrillator 12 may further include circuits (not shown) known in the art for monitoring a variety of physiological parameters of patient 10 such as blood pressure, blood oxygen saturation, body temperature, respiration rate, and the like.

Accordingly, output devices 34 may be used to display or otherwise present the values for these parameters measured by the circuits. The display screen or touch screen may also be used to present a pain assessment scale to the operator, in accordance with an embodiment of the invention. The pain assessment scale may be numerical or pictorial and may include an operator prompt and instructions regarding a preferred input method. The scale may also include a list of words relating to pain types and a prompt for an operator to input the selected word. Output device 34 may again prompt the operator to suggest possible courses of action, additional pain assessments, or provide further instructions based on the pain assessment entered via input device 30.

Input device 30 may include a keyboard, a touch screen, a button, a pointing device, a push knob, a soft-key, a switch, a voice recognition device, or the like. Input device 30 may be used to control the operation of defibrillator 12 and enter background information and pain assessment data for a patient. The pain assessment data, background information, and physiological data, along with any other electronic data gathered by defibrillator 12 or entered by the operator through input 30, may be compiled into an electronic report by processor 26, or by an external processing unit that obtains the data from defibrillator 12. The external processing unit may be a computer, e.g., at a hospital or clinic, to which data from defibrillator 12 is uploaded. Processor 26 or an external processing unit may generate trending information, to include in the report, based on the previously listed measurements taken over time.

In some embodiments, the report may take the form of an electronic run report that documents events during an emergency medical treatment or monitoring episode. The report incorporates pain assessment data received by input from a user of defibrillator 12, and physiological data representative of one or more physiological conditions of the patient. The pain assessment data may be input by the operator based on a pain assessment scale. The pain assessment data and the physiological data are stored in a memory, such as memory 36. The stored data, or a report prepared based on the data, may be transmitted into a hospital information system to become part of the patient's medical record.

Power source 32 generates energy to power processor 26 and, for those components that require power, input devices 30, output devices 34, and memory 36. Under the control of processor 26, charging circuit 28 transfers energy provided by power source 32 to energy storage circuit 24 for delivery as a defibrillation shock to patient 10. Charging circuit 28 comprises, for example, a flyback charger.

In addition to providing power for defibrillation shocks, and for processor 26, input device 30, output device 34, and memory 36, power source 32 may provide power for other components of defibrillator 12 not illustrated in FIG. 1, such as the physiological monitoring circuits that may be incorporated in the defibrillator as described above. It is understood that the voltage provided by power source 32 may be regulated as necessary for use by the components of defibrillator 12.

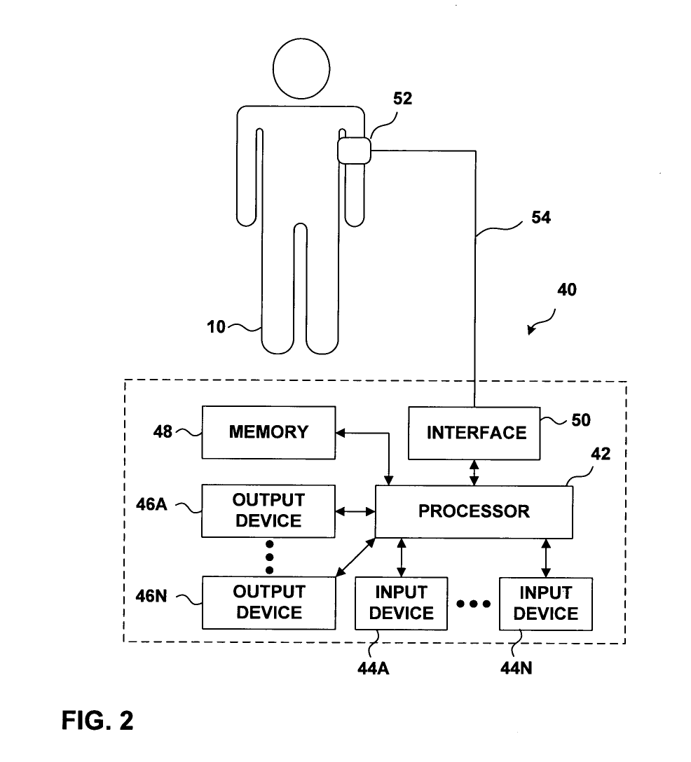

FIG. 2 is a block diagram illustrating an example patient monitor 40 as another embodiment of the invention. Patient monitor 40 is coupled to a patient 10 by a physiological condition detector, which may measure blood pressure, body temperature, cardiac rhythm, respiration rate, blood oxygen saturation, or the like. In FIG. 2, for the purposes of illustration, a physiological sensor 52 in the form of a blood pressure cuff 52 is placed around the arm of patient 10 to obtain a measurement of blood pressure. Conductor 54 couples blood pressure cuff 52 to patient monitor 40. Patient monitor 40 also includes a processor 42, one or more input devices 44A-44N (hereinafter 44), one or more output devices 46A-46N (hereinafter 46), a memory 48, and a physiological signal processing interface 50.

Conductor 54 is coupled to interface 50, and may include both a signal path and ground return. In a typical application, interface 50 includes a receptacle, and conductor 54 plugs into the receptacle. The physiological data received by interface 50 from a physiological sensor 52 in the form of a blood pressure cuff is sent to processor 42. Processor 42 may then generate an appropriate response to the received signal. In particular, processor 42 may store a digital representation of the signal in memory 48, convert the signal to a digital value, transmit an operator prompt to output 46, drive output device 46 to present a representation or indication of the signal, or the like.

Output device 46 may include a display screen, a touch screen, an indicator light, a speaker, or the like. Processor 42 may display instructions to an operator via the display screen or the touch screen, and the physiological condition of patient 10 monitored by blood pressure cuff 52 may also be displayed via the screens. As in the embodiment of defibrillator 12 described with reference to FIG. 1, the display screen or touch screen in patient monitor 40 may also be used to present a pain assessment scale to the operator. The pain assessment scale may be numerical or pictorial and may include an operator prompt and instructions regarding a preferred input method. The scale may also include a list of words relating to pain types and a prompt for an operator to input the selected word. Output device 46 may then prompt the operator, suggesting possible courses of action or additional pain assessments, or presenting further instructions based on the pain assessment entered via input 44 and/or the physiological data obtained via sensor 52.

Input 44 may include a keyboard, a touch screen, a button, a pointing device, a push knob, a soft-key, a switch, a voice recognition device, or the like. Input 44 may be used to control the operation of patient monitor 40 and enter a patient's background information and pain assessment data. The patient's pain assessment data and background information, along with the physiological data gathered by patient monitor 40 and any other electronic data entered by the operator through input 44, may be compiled into a report by processor 42 or an external processing unit. Again, as in the embodiment of FIG. 1, processor 42 of FIG. 2 or an external processing unit may generate trending information to include in the report, based on the previously listed measurements taken over time. The report may later be transmitted into a hospital information system to become part of the patient's medical record.

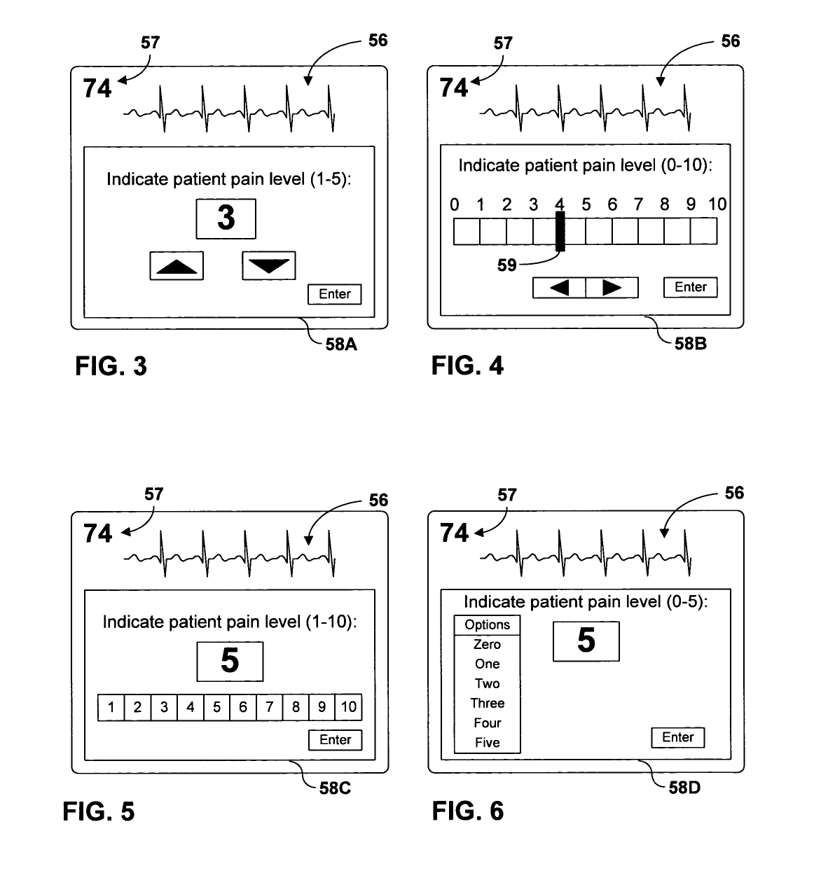

FIG. 3 through FIG. 9 are block diagrams illustrating various examples of defibrillator or patient monitor displays using a patient pain assessment user interface 58A-58G in accordance with the invention. FIG. 3 depicts an example of physiological data in the form of ECG data 56 and a numerical heart rate measurement 57 along with a pain assessment user interface 58A. Other physiological data may be presented, e.g., as an alternative to or in combination with ECG data 56. User interface 58A may present an operator with a pain assessment scale, a prompt asking for input, and input instructions if needed.

The patient pain assessment user interface 58A illustrated in FIG. 3 uses a numerical scale from 1 to 5 that can be entered by up and down arrow keys as displayed. In the example of FIG. 3, the “enter” key is pressed once the chosen pain assessment value is displayed on the screen. The input device may be arrow keys on a keyboard, soft-keys on a display, buttons, or the displayed arrows on a touch screen. The enter operation may be performed by any of the input options previously listed.

FIG. 4 illustrates a patient pain assessment user interface 58B that uses a numerical scale from 0 to 10 on a horizontal axis. The pain value is entered by right and left arrow keys, as shown in FIG. 4. In particular, the arrow keys serve to cause a sliding indicator 59 to change its position along the horizontal axis. Once indicator 59 is moved to the location of the chosen value, the enter key is pressed. Again, the input device may be arrow keys on a keyboard, soft-keys, buttons, or the displayed arrows on a touch screen.

FIG. 5 illustrates a patient pain assessment user interface 58C that uses a numerical scale from 1 to 10. This example also uses a horizontal axis similar to that shown in FIG. 4; however, buttons, not arrow keys, are used to enter the value in this case. In particular, the ten possible values are displayed on the screen along with the chosen value and a prompt to press the enter key once the choice has been made, as shown in FIG. 5. The input device may be number keys on a keyboard, soft-keys, buttons, or the displayed numbers on a touch screen.

FIG. 6 illustrates a patient pain assessment user interface 58D that uses a numerical scale from 0 to 5. The value options are presented in text form along a vertical axis on the screen and the selected value is displayed in numerical form, as shown in FIG. 6. The values may be selected with a pointing device or touch screen input. When the correct value is displayed, the enter key is pressed to input the information. As an alternative, the input device may be a voice recognition device. The enter operation may also be performed by any of the input options previously listed.

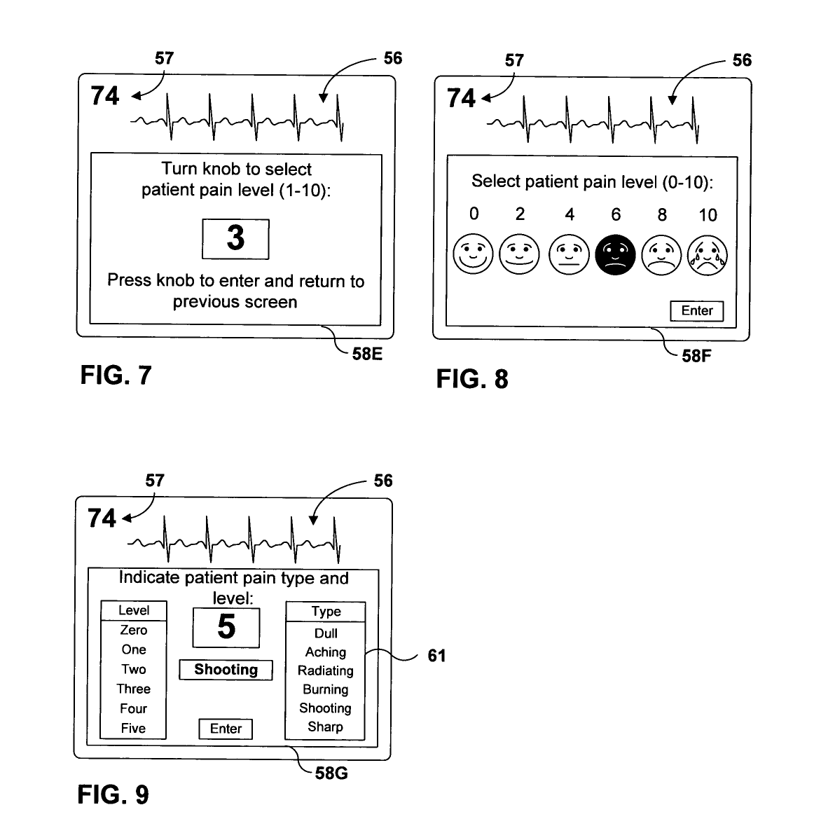

FIG. 7 illustrates a patient pain assessment user interface 58E that uses a numerical scale from 1 to 10. Instructions regarding the use of a knob to enter the pain assessment value are displayed on the screen along with the chosen value, as shown in FIG. 7. The input device in this example is a knob, mounted on the defibrillator or patient monitoring device, that may be turned until the value given by the patient appears on the display. In some embodiments, the knob is pushed inward to enter the displayed number.

FIG. 8 illustrates a patient pain assessment user interface 58F that uses a numerical scale from 0 to 10, in increments of two, along with pictorial representations for each value. The pictorial pain representations may be line drawings of facial expressions that correspond to the amount of pain associated with each number. The chosen facial expression is highlighted when indicated, as shown in FIG. 8. The input device may be number keys on a keyboard, arrow keys on a keyboard, soft-keys, buttons, a pointing device for a touch screen, or the facial expression pictures on a touch screen. Once the value is chosen and the facial expression is highlighted, the enter key may be pressed by any of the input devices listed.

FIG. 9 illustrates a patient pain assessment user interface 58G that uses a numerical scale from 0 to 5 along with a pain type scale 61. The pain value scale in FIG. 9 is similar to the scale shown in FIG. 6. The pain value options are presented in text form along a vertical axis on the screen and the pain type options are presented as descriptive words also along a vertical axis 61 on the screen. The addition of the pain type scale may further specify the pain a patient is feeling by using descriptive words including shooting, sharp, burning, radiating, aching, dull, and the like. The selected pain value is displayed in numerical form and the selected pain type is displayed in text form, as shown in FIG. 9. The options may be selected with a pointing device or touch screen input. When the correct pain value and type are displayed, the enter key is pressed to input the information. As an alternative, the input device may be a voice recognition device. The enter operation may also be performed by any of the input options previously listed.

In the various embodiments illustrated in FIGS. 3-9, input media such as buttons, keys, soft keys, touch screen keys, knobs and the like may be incorporated in the defibrillator or patient monitoring device. As an alternative, however, such input media may be incorporated in a remote control device, such as a handheld communication device carried by a medical technician or other device operator. In this case, the remote control device may include a wired or wireless transmitter and the defibrillator or patient monitoring device may include a wired or wireless receiver to receive pain assessment input or other instructions from the operator. The remote control device and defibrillator or monitoring device may communication in a variety of ways, e.g., radio frequency communication, infrared communication, and the like.

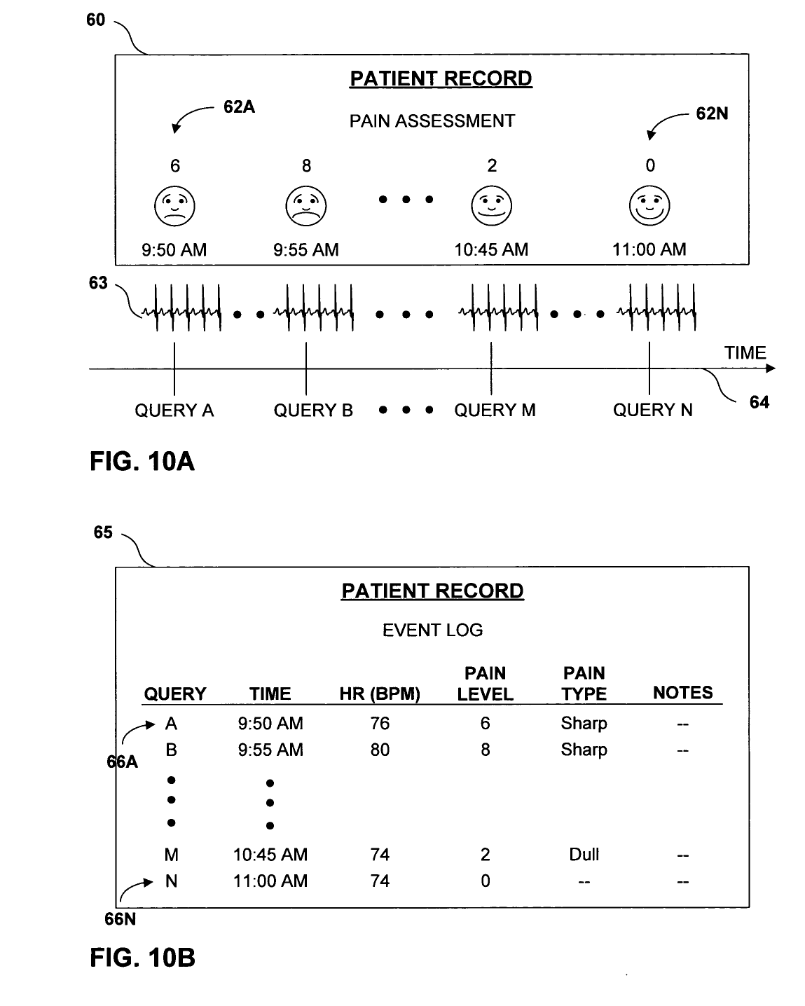

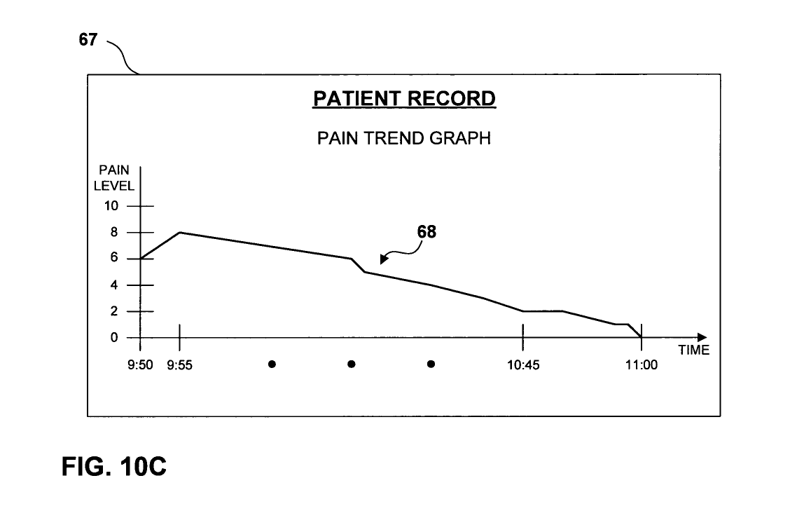

FIG. 10A is a block diagram conceptually illustrating a report 60 from an example patient record. The patient record shows a pain assessment report 60 that may be generated by data obtained via a patient pain assessment user interface in accordance with the invention. Pain assessment report 60 includes one or more entries 62A-62N (hereinafter 62) corresponding to one or more queries displayed over a horizontal time axis 64. When an operator takes a pain assessment from a patient, the input value is transmitted to a processor and then stored in a memory, as described in FIG. 1 and FIG. 2. When the pain assessment data is stored, the time is also recorded from an internal device, an external device, or operator entry. Each entry 62 includes the pain assessment value or a picture as shown in FIG. 10A to indicate the pain assessment, and the time at which the assessment was recorded.

Also recorded with entry 62 may be additional information from the operator such as notes about the condition of the patient and treatment being administered. In addition, report 60 may include physiological data obtained during the course of a treatment or monitoring episode. The physiological data and the pain assessment data may be synchronized to a common time line to enable temporal correlation of the pain assessments with particular physiological data such as blood pressure, blood oxygen saturation, body temperature, cardiac rhythm, respiration rate, and the like. In the example of FIG. 10A, an ECG 63 is shown in conjunction with pain assessment entries 62. Hence, the pain assessment data and physiological data may be used to later analyze a treatment or monitoring episode, e.g., for trending and diagnostic purposes, or simply for recordkeeping.

Pain assessment report 60 illustrates one example of trending information that may be generated by repeated pain assessments recorded over time. For example, report 60 enables an observer to readily ascertain whether the patient's pain is increasing or decreasing during the course of treatment or monitoring. Records of this type may help medical personnel decide what type of treatment is best for each patient. In some cases, the pain assessment trend information may permit development of historical data over several episodes of treatment or monitoring. The patient record may have additional pages for the patient's background information, test results, treatment decisions, physiological data, further trending information, and the like.

FIG. 10B is a block diagram conceptually illustrating another report from an example patient record. The patient record shows an event log 65 that may be generated by data obtained from a patient during the course of a treatment or monitoring episode. Event log 65 includes information similar to pain assessment report 60 shown in FIG. 10A, but in a different format and with additional information. Event log 65 includes one or more entries 66A-66N (hereinafter 66) corresponding to one or more queries displayed along a vertical axis. Each entry 66 includes the time at which the data was recorded, a heart rate value or other physiological data value, a patient pain level value, a patient pain type value, and any relevant notes input by an operator.

Event log 65 may record all actions performed by the operator relating to the patient pain assessment user interface and the defibrillator or patient monitor it may be incorporated in. Records of this type may help medical personnel follow the exact steps taken to treat a patient and may allow an easier transition between emergency staff members and subsequent care givers.

FIG. 10C is a block diagram conceptually illustrating another report from an example patient record. The patient record shows a pain trend graph 67 that may be generated by data obtained via a patient pain assessment user interface in accordance with the invention. Pain trend graph 67 plots the numerical pain levels recorded in pain assessment report 60, shown in FIG. 10A, dependent upon the time at which the data was recorded.

Pain trend graph 67 illustrates another example of trending information that may be generated by repeated pain assessments recorded over time. For example, graph 67 enables an observer to view a pain trend line 68 of the entire course of treatment or monitoring episode, and not just discrete pain values as in pain assessment report 60 shown in FIG. 10A. Using pain trend line 68, hospital personnel may easily determine how quickly a treatment relieved the patient's pain and at what time the most significant drops in pain level occurred.

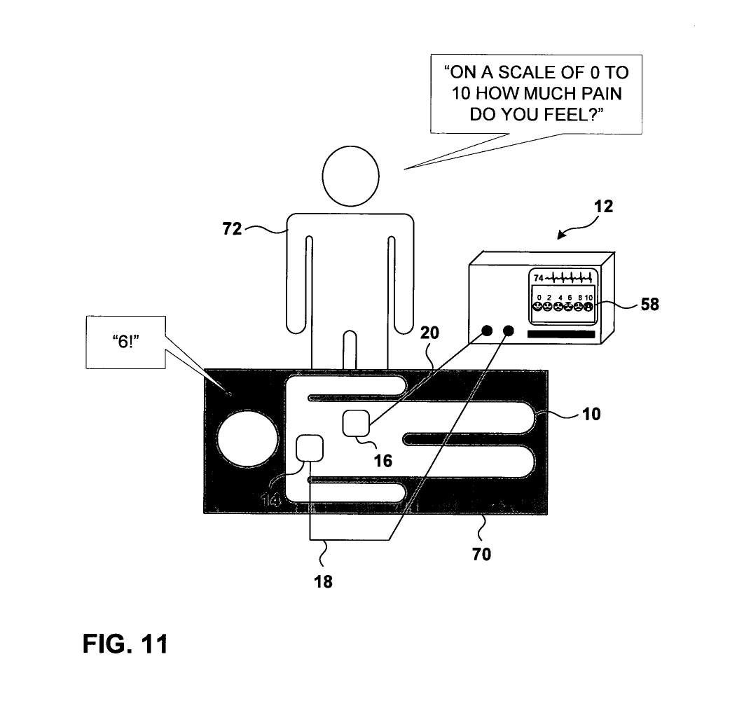

FIG. 11 is a block diagram illustrating an example interaction between a patient 10 and a defibrillator operator 72. In the example of FIG. 11, patient 10 is lying on a gurney 70, and is attached to a defibrillator 12 via an electrode 14 and an electrode 16. Electrodes 14 and 16 are coupled to defibrillator 12 by a conductor 18 and a conductor 20 respectively, as shown in FIG. 1 and FIG. 10. Defibrillator 12 includes an example of a patient pain assessment user interface 58 similar to the one shown in FIG. 8. Defibrillator operator 72 asks patient 10, “On a scale of 0 to 10 how much pain do you feel?” Patient 10 responds by saying, “6!” Defibrillator operator 72 may read the pain assessment scale aloud to patient 10 because patient 10 may be unable to view the pain assessment scale. Patient 10 may also read the pain assessment scale directly from pain assessment user interface 58 or, in this case, determine the pain value by examining the pictorial representations.

Defibrillator operator 72 may then enter the pain assessment value given by patient 10 into defibrillator 12 by an input device as described in FIG. 8. The original pain assessment may be completed at any time, as long as the patient is capable of answering. This may delay the initial pain assessment until after defibrillation, if needed. Defibrillator 12 may then issue a prompt to the operator based on the pain assessment value entered. This prompt may suggest future treatment or ask for another pain assessment.

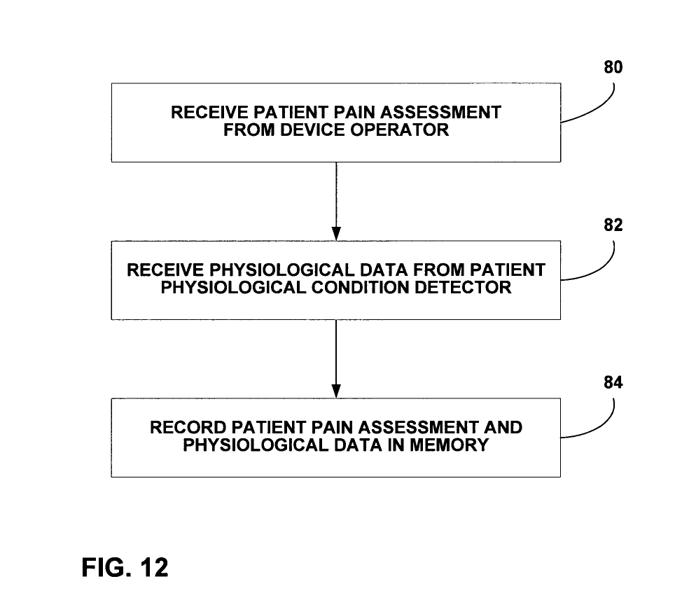

FIG. 12 is a flow chart illustrating a method for using a pain assessment user interface 58. A defibrillator processor 26 or a patient monitor processor 42 (hereinafter the processor) first receives a patient pain assessment from the operator (80). The processor also receives physiological data from a patient physiological condition through a defibrillator 12 or a patient monitor 40 (hereinafter the device) (82). The patient pain assessment and physiological data received are both recorded in a defibrillator memory 36 or a patient monitor memory 48 (hereinafter the memory) by the processor (84). The steps listed in FIG. 12 outline the core functions the processor performs to operate the pain assessment user interface 58.

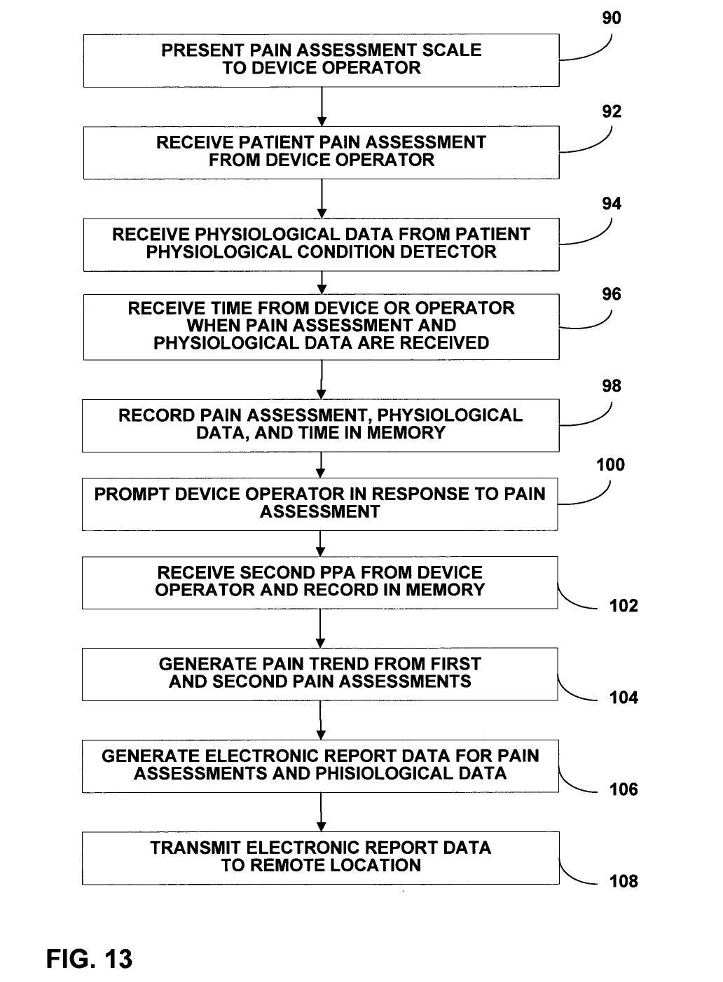

FIG. 13 is a flow chart illustrating a method for using a patient pain assessment user interface 58 in greater detail. The processor first presents a pain assessment scale to an operator (90). The presentation may include a prompt for the operator to enter a pain assessment value. The prompt may include instructions for a preferred input process of the pain assessment. The patient pain assessment value is then received from an input device used by the operator (92). Physiological data is also received from one or more patient physiological condition detectors included in the device (94). When the patient pain assessment and the physiological data are received by the processor, the time is also received (96). The time may be determined by an internal device, an external device, or operator input. The processor then records the patient pain assessment, physiological data, and time into the memory of the device (98).

The processor may generate another operator prompt in response to the patient pain assessment (100). This prompt may simply acknowledge receiving the pain assessment or may give a suggestion for further treatment or further pain assessments. Treatment suggestion prompts may depend on how much pain the patient reports to be experiencing and the physiological data received. A prompt for additional pain assessments may be given after a set period of time, when physiological data reaches a specified level, when medication levels reach a predetermined point, if the operator notes a procedure on the device, if the device receives a positive indication, if an error occurs in transmitting the original assessment, or the like. The operator may note the use of such drugs as nitroglycerin or morphine as a patient therapy on the pain assessment user interface 58. The patient pain assessment user interface 58 may then default to prompting for a pain assessment every 5 minutes. The device may also receive a physiological condition signal, such as positive changes in a diagnostic ECG (12-lead) indicating an acute myocardial infarction, that may require an additional pain assessment.

The patient pain assessment user interface 58 may prompt the device operator for a second patient pain assessment due to an elapsed amount of time or an event triggered by the device or the operator, as discussed above. The operator queries patient 10 again and enters the reported pain level. This patient pain assessment is recorded in the memory again along with the time at which the measurement was received (102). Physiological data generally constitutes a continuous measurement, but if data only needs to be measured periodically, it may be collected at the same time as the second patient pain assessment.

The processor or an external processing unit may then generate trending information for the pain assessment values gathered over time (104), as is typically done for physiological data. A report may be generated by the processor or the external processing unit. The report generally includes all the patient pain assessment values and physiological data and the times at which they were recorded (106). The report may also include any trending information generated and the patient's background information such as name, age, sex, and the like. The report may then be transmitted to a remote location (108), most likely a hospital patient database.

The transmission may be done via a wired or wireless connection, depending on the device and its preferred function. In some cases, the entire report may be generated by a processor within the defibrillator or monitoring device. In other instances, the defibrillator or monitoring device may store pain assessment and physiological data for upload to an external processing unit, such as a computer, for generation of the report. Thus, a report generation computer may operate as an intermediary between the defibrillator or monitoring device and a hospital medical information database. The report may become part of the patient's medical record with the pain assessment values included electronically along with the physiological data trends.

Including pain assessment values and trends in an electronic medical record may increase the use of pain assessment in diagnosis and treatment of patients. The invention may also help medical staff members understand the amount of pain a patient is experiencing to ensure the patient is comfortable.

Various embodiments of the invention have been described in the above figures.

These and other embodiments are within the scope of the following claims.

1. A method comprising:
receiving a patient pain assessment from an operator, wherein the patient pain assessment is based on a pain assessment scale;
receiving physiological data representative of a patient physiological condition; and
recording the patient pain assessment and the physiological data in a memory.

2. The method of claim 1, further comprising generating electronic report data incorporating both the patient pain assessment and the physiological data.

3. The method of claim 2, further comprising transmitting the electronic report data to a remote location.

4. The method of claim 1, further comprising recording a time when the patient pain assessment and the physiological data are received.

5. The method of claim 4, further comprising receiving the time from one of an internal device, an external device, and an operator interface.

6. The method of claim 1, wherein the patient pain assessment is a first patient pain assessment; the method further comprising:
receiving a second patient pain assessment from the operator; and
recording the second patient pain assessment in memory.

7. The method of claim 6, wherein the second patient pain assessment is received subsequent to the first patient pain assessment.

8. The method of claim 6, further comprising generating a pain trend as a function of the first and second patient pain assessments.

9. The method of claim 1, further comprising prompting the operator in response to the patient pain assessment.

10. The method of claim 1, further comprising presenting the pain assessment scale to the operator.

11. The method of claim 10, wherein presenting the pain assessment scale comprises at least one of presenting a numerical pain assessment scale and presenting a pictorial pain assessment scale.

12. The method of claim 10, wherein presenting the pain assessment scale comprises presenting a pain type scale.

13. The method of claim 12, wherein the pain type scale comprises words descriptive of a type of pain sensation; the words further comprising shooting, sharp, burning, radiating, aching, and dull.

14. The method of claim 1, wherein the patient physiological condition includes one of blood pressure, blood oxygen saturation, body temperature, cardiac rhythm, and respiration rate.

15. A device comprising:
a first input device to receive a patient pain assessment from an operator, wherein the patient pain assessment is based on a pain assessment scale;
a second input device to receive physiological data representative of a patient physiological condition; and
a memory to record the patient pain assessment and the physiological data.

16. The device of claim 15, wherein the device comprises an external defibrillator.

17. The device of claim 15, wherein the device comprises an external patient monitor.

18. The device of claim 15, wherein the first input device comprises at least one of a keyboard, a soft-key, a button, a touch screen, a pointing device, a push knob, and a voice recognition device.

19. The device of claim 15, wherein the second input device comprises at least one of a keyboard, a voice recognition device, a touch screen, a pointing device, and a patient physiological condition detector.

20. The device of claim 15, wherein the first input device and the second input device are the same device.

21. The device of claim 15, further comprising an output device to present to the operator at least one of a pain assessment scale and a prompt in response to a patient pain assessment.

22. The device of claim 21, wherein the output device comprises at least one of a display screen, a touch screen, an indicator light, and a speaker.

23. A computer-readable medium comprising instructions to cause a processor to:
receive a patient pain assessment from a user interface, wherein the patient pain assessment is based on a pain assessment scale;
receive physiological data representative of a patient physiological condition; and
record the patient pain assessment and the physiological data within a memory.

24. The computer-readable medium of claim 23, further comprising instructions that cause the processor to generate electronic report data incorporating both the patient pain assessment and the physiological data.

25. The computer-readable medium of claim 24, further comprising instructions that cause the processor to transmit the electronic report data to a remote location.

26. The computer-readable medium of claim 23, further comprising instructions that cause the processor to record a time when the patient pain assessment and the physiological data are recorded.

27. The computer-readable medium of claim 26, further comprising instructions that cause the processor to receive the time from one of an internal device, an external device, and an operator interface.

28. The computer-readable medium of claim 23, wherein the patient pain assessment is a first patient pain assessment, the computer readable medium further comprising instructions that cause the processor to:
receive a second patient pain assessment from the operator; and
record the second patient pain assessment in memory.

29. The computer-readable medium of claim 23, further comprising instructions that cause the processor to transmit a signal to an output device to prompt the operator in response to the patient pain assessment.

30. The computer-readable medium of claim 23, further comprising instructions that cause the processor to transmit the pain assessment scale to an output device to present the pain assessment scale to the operator.
